(19) United States
(12) Patent Application Publication
(10) Pub. No.: US 2012/0277648 A1
(43) Pub. Date: Nov. 1, 2012

(54) Adhesive bandage with raised portion

(76) Inventor: Tianna Michelle Kendall, Pullman, WA (US)

(21) Appl. No.: 13/066,929

(22) Filed: Apr. 28, 2011

(51) Int. Cl. A61F 13/02

(52) U.S. Cl. 602/55

(57) ABSTRACT

Adhesive bandages with at least one raised non-adhesive portion which is elevated above the at least one adhesive layer. The raised non-adhesive portion is peripherally surrounded by an adhesive layer and is designed to protect surgical sites and cutaneous wounds as well as any other skin malady. Neither the raised non-adhesive portions of the bandage nor the adhesive layer are in contact with the cutaneous layer which is the subject of the sought protection, with the adhesive layer coming into contact only with the area surrounding the affected site which can result in the reduction or elimination of exposure to moisture and/or contaminates. In addition, the surgical site or cutaneous wound is protected from force or impact. The raised non-adhesive portion may also be transparent to provide for monitoring of the surgical site or cutaneous wound while the surgical site or cutaneous wound remains protected.

### BACKGROUND

Throughout the lengthy history of medicine, a common and serious problem has been discovering new and improved ways of keeping wounds, cutaneous surgical sites and other cutaneous maladies clean and dry in order to facilitate rapid healing. This is especially difficult when some aspect of the wound is raised in such a way that traditional bandages cannot adequately cover it in any type of sealed way, and/or the elevated plane and differing surfaces of some wounds raise the bandage unevenly which can allow air and water to potentially make contact with the wound. In addition, the strip shape of traditional bandages does not offer any sort of seal or added protection along all the edges, particularly, as is the case in some traditional bandages which have gauze in the central portion of the inner face of the bandage.

This difficulty arises, for example, in the case of newborn babies, where the remaining umbilical stump resulting from the severing of the umbilical cord is extremely delicate and prone to infection. Medical professionals advise to keep this umbilical remnant clean and dry to facilitate its eventual healing and subsequent dropping off. Keeping a newborn clean and dry is already challenging, especially with regard to the stump which is in close proximity to body parts which need to be washed regularly to avoid other localized infection. In this way, there is a need for a solution which can keep the umbilical stump protected and dry during such activities such as bathing, which at the same time does not come into contact with the stump which can result in discomfort and possible irritation. Traditional bandages are simply not designed for such tasks.

An additional cutaneous issue is trying to keep peripherally inserted central catheter lines (hereinafter, “PICC lines”) and other medical apparatuses such as accessed ports dry while at the same time allowing the patient to live a normal life in which bathing and occasional exposure to moisture is an unavoidable necessity. Traditional adhesive bandages also fail in this regard as the adhesive is often not water resistant and in addition, the issue of a raised surface, previously discussed, makes it difficult to have any full coverage over an uneven area without the possibility of resulting air pockets gapping and subsequent potential introduction of moisture, particularly when there is no protective adhesive layer surrounding every edge of the bandage. In addition, traditional bandages have continuous contact with the skin which can further irritate the area of contact with the bandage and prolong healing time, not to mention causing pain to the area with the potential of constant rubbing and/or contact. Along the same lines, removal of traditional bandages from cutaneous wounds or surgical sites can be extremely painful as there is a potential that the adhesive portion will have adhered to at least part of the cutaneous wound or surgical site.

The same challenge of keeping an area clean and dry is also inherent in cases where there are sutures or stitches. One is often advised that such areas be kept dry which is challenging in one's day to day life. Again, traditional bandages which often have an adhesive layer which is located close to the wound site can stick to the wound resulting in discomfort and in other cases the possibility of accidental pulling and/or removal of the stitches or sutures.

Traditional bandages also do not allow for monitoring of the wound site. Once a traditional bandage is applied, the cutaneous wound or surgical site beneath it is not visible as it is covered by the bandage. The unknown state of the cutaneous wound or surgical site can cause a great deal of stress and anxiety, especially in cases in which it is imperative to keep such sites completely clean and dry. In the course of bathing or other exposure, one is not able to determine if the bandage is actually successful in keeping the wound clean and dry. This introduces the real and dangerous' possibility of wounds being subjected and/or exposed to prolonged moisture contact which is potentially extremely damaging. In the alternative, if one is able to monitor the wound site, one is able to immediately discontinue contact with moisture should there be even an infinitesimal amount of leakage which reduces the amount of damage which may result from prolonged exposure.

A further longstanding problem with traditional bandages relates to cutaneous wound and surgical site protection. Traditional bandages are generally formed completely with soft and flexible material. This material is usually lightweight and is not capable of protecting the cutaneous wound or surgical site from any external force. In this way, traditional bandages expose the covered area to potential harm from force which can be extremely damaging to the cutaneous wound or surgical site as well as being painful and prolonging the healing process.

### SUMMARY

In accordance with the following disclosure, the problem of cutaneous wound or surgical site exposure and irritation can be avoided by a bandage with a raised non-adhesive portion which is essentially peripherally surrounded by a partially adhesive layer. The partially adhesive layer can be formed into one or many tabs which can result in various different shapes of the bandage which enhances the ease by which the bandage is applied and removed, while also greatly reducing the possibility of skin puckering beneath the bandage. The inclusion or exclusion of tabs also allows for customization of the bandage and allows the bandage to better form to fit different body parts, which in turn decreases the possibility of gapping and/or lifting as a result of movement. The bandage can be circular or ovular in shape with no tabs and can also be water resistant. With regard to the appearance of the bandage, it can be all or partially transparent as a means by which the covered area can be continuously monitored.

### BRIEF DESCRIPTION OF THE DRAWINGS

FIG. 1 is an environmental view depicting an application of a bandage according to the accompanying disclosure.

FIG. 2 is a side view of a bandage in accordance with the current disclosure, depicted in a first exemplary application of use.

FIG. 3 is a side view of a bandage in accordance with the current disclosure, depicted in a second exemplary application of use.

FIG. 4 is a bandage in accordance with the current disclosure.

FIG. 5 is a plan view of the bandage depicted in FIG. 1.

FIG. 6 is a plan view of an alternative embodiment of the bandage depicted in FIG. 1.

FIG. 7 is a plan view of a further alternative embodiment of the bandage depicted in FIG. 1.

FIG. 8 is a side view of one embodiment of a bandage in accordance with the accompanying disclosure.

FIG. 9 is a side view of another embodiment of a bandage in accordance with the accompanying disclosure.

### DETAILED DESCRIPTION

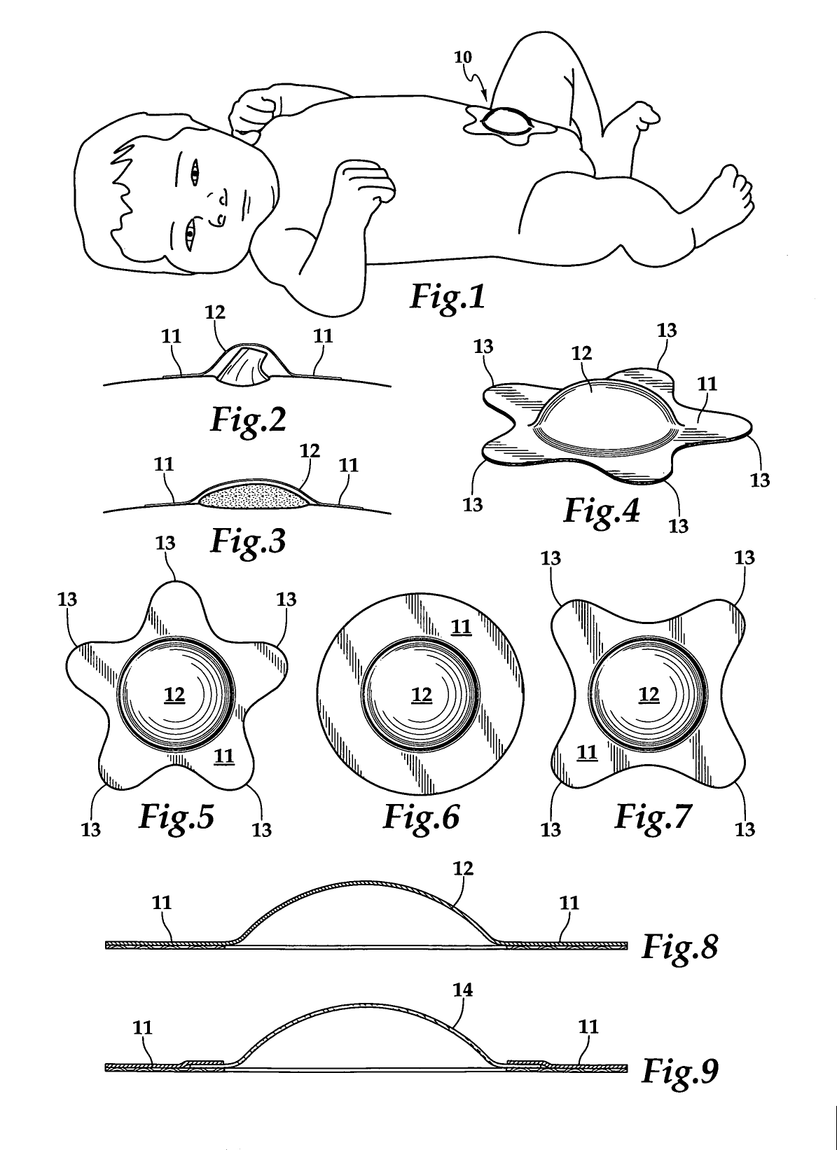

FIG. 1 depicts a preferred bandage 100 in accordance with the current disclosure in one possible field of use. In this particular embodiment, the bandage 100 is depicted as being used as a protective layer which covers an umbilical stump 5, FIG. 2, of a newborn. The protective layer can be water resistant. For example, the umbilical stump 5, FIG. 2, of a newborn baby is very delicate and must remain intact (with the infant) until the stump 5, FIG. 2, naturally drops off. This manner of use of the bandage 100 can be utilized as a means of protecting the stump 5, FIG. 2 during such activities which may cause potential harm to the umbilical stump 5, FIG. 2, such as bathing of the newborn.

FIG. 2 depicts a side view of a bandage 100 with adhesive layer 111 and raised non-adhesive portion 112. FIG. 2 depicts the bandage 100 in a field of use covering an umbilical stump 5. In this embodiment, the raised non-adhesive portion 112 is shown as transparent with the purpose of being able to view the stump S and monitor for leakage during activities such as bathing, but it need not be transparent and may also be opaque. Umbilical stumps 5 are extremely sensitive and delicate and the raised non-adhesive portion 112 is not in contact with the stump as to avoid irritation and discomfort. In addition, the raised non-adhesive portion 112 makes removal of the bandage 100 much easier and less dangerous as there is a greatly reduced chance that the slightly adhesive portion will come into contact with the sensitive stump which could potentially result in the bandage sticking to the stump 5 and pulling painfully during removal which is a common problem with prior art bandages in which the non-adhesive area lies flat against the skin and is not elevated above the adhesive portion which surrounds it.

FIG. 3 depicts the bandage 100 of FIGS. 1 and 2 in another possible field of use with the adhesive layer 111 and raised non-adhesive portion 112. FIG. 3 depicts the bandage 100 covering a raised cutaneous wound 6, although the bandage can be used to cover or protect any other cutaneous surface malady as well as medical apparatuses (including but not limited to PICC lines, accessed ports, sutures, stitches and other surgical sites), in the same way. In the embodiment depicted in FIG. 3, raised non-adhesive portion 112 protects the cutaneous wound 6 from exposure to moisture and other contaminates. The adhesive layer 111 can also be water resistant. The raised non-adhesive portion 112 can also be transparent to allow for monitoring of the site. Again, as is depicted in FIG. 2, the raised non-adhesive portion 112 is not in contact with the wound 6 in order to avoid irritation and discomfort which is different from prior art bandages in which the non-adhesive gauze is not raised above the adhesive layer. In addition, because the bandage 100 has no contact with the cutaneous wound site 6, and the raised portion 112 is non-adhesive there is a greatly decreased risk of the raised portion of the bandage 100 adhering to the wound 6 during removal of the bandage. Not only does this feature decrease the chances of the pain and irritation to a patient which could result from having to remove an adhesive layer from a wound 6, but it also decreases the risk of an adhesive layer coming into contact with sutures or stitches and inadvertently ripping them out during the removal process (ripping out stitches or sutures during the healing process can not only be painful, but potentially dangerous as well and the raised non-adhesive portion also decreases the likelihood of this occurrence).

FIG. 4 depicts the bandage 100 in FIGS. 1-5 and 8 with the adhesive layer 111 and a raised non-adhesive portion 112, both of which can be formed from any durable and flexible material including but not limited to silicone, rubber, or fabric all of which can be water resistant. In the preferred embodiment, the adhesive layer 111 is completely peripherally affixed (by adhesive or other method) to the rim of the base of the raised non-adhesive portion 112 which can be dome shaped; the adhesive layer 111 extending outward from the periphery of the rim of the base of the raised non-adhesive portion 112 such that the adhesive layer 111 is absent underneath the raised non-adhesive portion 112, such that neither the adhesive layer 111 nor the raised non-adhesive portion 112 come into contact with the cutaneous wound 6, FIG. 3, or umbilical stump, 5, FIG. 2, while the adhesive layer 111 extends outward from the edges of the base of the raised non-adhesive portion 112 and adheres to the skin surrounding the raised non-adhesive portion 112, securing the bandage 100 into place.

Additionally, in the embodiment depicted in FIG. 4, several tabs 113 are shown. For example, the adhesive layer 111 can be shaped into one or several tabs 113. These tabs 113 contribute to the ease of application and removal of the adhesive layer 111. The tabs 113 depicted in FIG. 4 are rounded, however this need not be the case and the tabs can be presented in several different shapes. FIG. 4 shows five tabs 113, however there can be more or fewer tabs which can be either uniform in shape or variable in shape. The tabs 113 also serve another important purpose in that they allow for more ease of movement of the area, onto which the bandage 100 is applied. For example, it is ordinarily desirable for living beings to move and the addition of one or several tabs creates a bandage which has enhanced flexibility over a bandage of the prior art, which tends to gap, especially in the center (i.e. over a wound 6, FIG. 3, or an umbilical stump 5, FIG. 2) with different types of movement. This gapping is undesirable as it exposes the wound site to unwanted moisture and other elements including contaminates. Providing tabs 113, the number of which can be chosen to best accommodate the region of the body to which the bandage is applied, can greatly customize the fit of the bandage 100 to its particular intended application, thereby substantially reducing the risk of gapping and the resulting risk of exposure. The raised non-adhesive portion 112 is again depicted and is virtually completely peripherally bordered by the adhesive layer 111. Depending on its fabrication, the adhesive layer 111 may also be re-adherable such that it can be reused many times, which is especially useful in cases in which it is only adhered for a short period of time such as during bathing.

FIG. 5 depicts the bandage 100 of FIGS. 1 and 4 including the adhesive layer 111 and the raised non-adhesive portion 112. In this embodiment, the adhesive layer, 111 is again depicted as being shaped into several tabs 113 which aid in easing the process of removing the adhesive layer. In addition, the multiple tabs 113 allow the bandage 100 to be applied in such a way as to adhere more continuously with the body of a patient, so as to greatly reduce the chance of under bandage skin puckering (which can lead to discomfort and wound leakage).

The tabs 113 also provide the potential for shapes which can be appropriate and popular with children. The bandage 100 of FIG. 5 also depicts that the raised non-adhesive layer 112 is surrounded entirely by the adhesive layer 111, which borders the raised portion 112 completely. For example, with respect to the bandage 100 of FIG. 5, there is no part of the raised non-adhesive portion 112 which creates the edge of the bandage. Encircling the elevated non-adhesive portion 112 with the adhesive layer 111 provides heightened structural security for the bandage 100. There is essentially not one portion of the raised non-adhesive portion 112 which is not anchored by the adhesive layer 111. The anchoring by the adhesive layer 111 greatly reduces the chances of lifting of the bandage 100 or gapping of the bandage 100 which can result in the wound site 6, FIG. 3 or umbilical stump, 5 FIG. 2, being exposed to unwanted elements (such as moisture or contaminates, which are a prevalent problem with prior art strip shaped bandages having edges which are not anchored by any adhesive).

FIG. 6 depicts a bandage 200 with an adhesive layer 211 and raised non-adhesive layer 212. In this embodiment, the adhesive layer 211 is rounded and has no angular edges. Oftentimes, with cutaneous wounds 6, FIG. 3 or umbilical stumps 5, FIG. 2, the surrounding area is sensitive. This embodiment is devoid of any tabs resulting in an alternate mode of removal as only one rounded edge needs to be peeled upward in order to facilitate the removal of the bandage in its entirety which reduces the potential irritation which may be caused by having to peel or lift the bandage at multiple sites. In this embodiment, the raised non-adhesive portion 212 can be dome shaped and peripherally attached at the rim of its base to the adhesive layer 211 which extends radially outward from the raised non-adhesive portion 212. Neither the raised non-adhesive portion 212 which is raised above the adhesive layer 211, nor the adhesive layer are in contact with the wound 6, FIG. 3, or umbilical stump 5, FIG. 2 which the bandage 200 covers. Both the raised non-adhesive portion 212 and the adhesive layer 211 may be formed from any polymer or other material which can be flexible and water resistant. The raised non-adhesive portion 212 and the adhesive layer 211 need not be made from the same material and the raised non-adhesive portion 212 may be reinforced in order to make it thicker than the adhesive layer 211.

FIG. 7 depicts a bandage 300 with partially adhesive layer 311 and raised non-adhesive portion 312. In this embodiment, the adhesive layer. 311 is again shaped into separate tabs 313 for easy application and removal. FIG. 7 depicts four such tabs 313 although again, there may be a greater number of tabs or a lesser number thereof. For example, as was discussed earlier, the bandage 100, 300 is highly customizable with regard to the addition of tabs 113, 313 or as in FIG. 6, the absence of any tabs. This customizable nature results in the adhesive bandage (100, 200, 300) being capable of securely covering various different types of wounds 6, FIG. 3, and umbilical stumps 5, FIG. 2, in addition to being able to be tailored to best fit a plethora of different body parts. For example, a bandage which is shaped to fit perfectly on an elbow might not be the same shape as a bandage which is shaped to fit perfectly on a forehead. Again, the raised non-adhesive portion 112, 212, 312 is depicted completely, peripherally surrounded by the adhesive layer (respectively 111, 211, 311).

FIG. 8 depicts a side view of the bandage 100, FIGS. 1-5 and 8 with adhesive layer 111 and raised non-adhesive portion 112. FIG. 8 depicts the raised non-adhesive portion 112 which allows for discontinuous contact of the bandage 100 with the cutaneous wound 6, FIG. 3 or umbilical stump 5, FIG. 2, which results in a decreased chance of irritation or rubbing to the cutaneous wound 6, FIG. 3 or umbilical stump 5, FIG. 2. FIG. 8 show the adhesive layer 111 peripherally attached to the edges of the raised non-adhesive portion 112 such that the raised non-adhesive portion 112 forms a dome shape which has no contact with a wound 6, FIG. 3 or umbilical stump 5, FIG. 2 beneath it and is secured by the adhesive layer 111 which extends outward from the raised non-adhesive portion 112.

FIG. 9 depicts another side view of a bandage 400, with adhesive layer 411 with a rigid or more resistant raised portion 414. In this embodiment, the raised non-adhesive layer 412 is comprised of a rigid or non-flexible material such as plastic or other polymer which protects the wound from stronger forces or impacts as well as protecting the wound 6, FIG. 3, or umbilical stump 5, FIG. 2, from exposure to moisture or other contaminates. In this embodiment, the raised non-adhesive layer 412 is cup shaped. The adhesive layer 411 can be permanently affixed peripherally at the rim of its base by an adhesive or other means. The adhesive layer 411 extends outward from the base of the raised non-adhesive portion 412 and the adhesive layer 411 adheres to the skin surrounding the wound 6, FIG. 3 or umbilical stump 5, FIG. 2 site. Neither the raised non-adhesive portion 412, nor the adhesive layer 411 are in contact with the wound 6, FIG. 3, or umbilical stump 5, FIG. 2. As in the previous embodiments, the rigid raised portion 414 is completely peripherally surrounded by the adhesive layer 411 and can also have no tabs or several tabs. In addition, the rigid raised portion 414 can be transparent or opaque.

I claim:

1. A bandage comprising:
at least one raised non-adhesive portion having at least one rim; and
at least one adhesive layer;
wherein said at least one adhesive layer is peripherally affixed to said at least one rim of said at least one non-adhesive portion; and
wherein said at least one raised non-adhesive portion is raised above said at least one adhesive layer.

2. The bandage of claim 1, wherein said at least one raised non-adhesive portion is dome shaped.

3. The bandage of claim 1, wherein said at least one adhesive layer is shaped into at least one tab.

4. The bandage of claim 1, wherein the entirety of said bandage is water resistant.

5. The bandage of claim 1, wherein said at least one raised non-adhesive portion is transparent.

6. The bandage of claim 1, wherein said at least one adhesive layer is transparent.

7. The bandage of claim 1, wherein said at least one adhesive layer is formed from a flexible material.

8. The bandage of claim 1, wherein said raised non-adhesive portion is formed from a flexible material.

9. The bandage of claim 1, wherein said at least one raised non-adhesive portion is formed from a rigid material.

10. The bandage of claim 1, wherein said at least one adhesive layer is re-adherable.
